(19) United States
(12) Patent Application Publication
(10) Pub. No.: US 2003/0109548 A1
(43) Pub. Date: Jun. 12, 2003

(54) Compositions and methods of treating iron excess

(76) Inventors: Paulette W. Royt, Fairfax, VA (US); Robert V. Honeychuck, Fairfax, VA (US); Wayne M. Stalick, Fairfax, VA (US)

Correspondence Address: Jennifer Murphy, Director, George Mason University, Office of Technology Transfer, 4400 University Drive, MSN 3A2, Fairfax, VA, 22030, US

(21) Appl. No.: 10/289,392

(22) Filed: Nov. 7, 2002

Related U.S. Application Data
(60) Provisional application No. 60/331,199, filed on Nov. 9, 2001.

(51) Int. Cl. A61K 31/47; A61K 9/127

(52) U.S. Cl. 514/312; 424/450

(57) ABSTRACT

The present invention relates to compositions comprising 4-hydroxy-2-alkylquniolines for depleting liquids of iron, and for treating diseases associated with iron overload, toxicity, or iron excess. The 4-hydroxy-2-alkylquniolines can be associated with any material in which it retains its iron-binding properties.

This application claims the benefit of U.S. Provisional Application No. 60/331,199, filed Nov. 9, 2001, which is hereby incorporated by reference its entirety.

### BACKGROUND OF THE INVENTION

Microorganisms synthesize a variety of iron chelators that are secreted by the cells and serve to solubilize external iron before transport into the cell. Called siderophores, these ligands typically form six-coordinate octahedral complexes with iron. Catecholates, phenolates, and lo hydroxamates are chelating groups most often identified in microbial siderophores. However, novel functional binding groups including oxazoline nitrogen, alpha-hydroxy carboxylates, complexone-like structures, and hydrazide have more recently been identified in microbial siderophores (Matzanke, B. F. 199, p. 15-60. In G. Winkelmann (ed.), CRC Handbook of Microbial Iron Chelators, CRC Press. Boca Raton, Fla.). Some microorganisms use siderophores to store iron following entry into the cell (Ratledge, C. 1987, p. 207-221. In Winkelmann, G., D. van der Helm, and J. B. Nielands (ed.), Iron Transport in Microbes, Plants and Animals. VCH, Weinheim), protecting the cell from oxidative damage caused by free iron. Bacteria also use haem-containing bacterioferritins and haem-free ferritins to store iron. These proteins have a hollow core in which up to 2000 iron atoms can be stored as ferric-hydroxyphosphate (Andrews, S. C. 1998, p. 281-351. In A. H. Rose and J. F. Wilkinson, (ed.), Advances in Microbial Physiology,Vol. 4. Academic Press, New York).

A study of iron transport and metabolism in Pseudomonas aeruginosa led to the isolation of an iron chelator associated with the cytoplasmic membrane of iron-rich cells (Royt,1988. Biochim. Biophys. Acta, 939: 493-502). Known as the membrane associated iron chelator (MAIC), the compound is extracted from membranes with ethanol, and in earlier studies, isolated on thin-layer chromatograms. These chromatograms revealed a purple band and a brown band, both of which contained bound Fe. This was evident from radiolabeled experiments in which ⁵⁵FeCl₃ was added to the growth medium. The ethanol extract of membranes of these cells revealed radiolabeled brown and purple bands on TLC plates. Also, addition of ⁵⁵FeCl₃ to the complete ethanol extract of cells resulted in the appearance of radiolabeled bands on a thin layer chromatogram. A role of the chelator in iron transport or in iron storage was suggested.

### DESCRIPTION OF THE DRAWINGS

FIG. 1 shows the structure of 4-hydroxy-2-nonylquinoline.

### DESCRIPTION OF THE INVENTION

It has now been shown that the purple and brown bands are two forms of the chelator. The compounds have been purified from the ethanol extract of iron-rich P. aeruginosa by high-pressure liquid chromatography (HPLC). The structure of the brown compound, determined by NMR and FAB mass spectroscopy, is that of 4-hydroxy-2-nonylquinoline. The purple compound has been identified as 4-hydroxy-2-heptylquinoline. Previously isolated from the filtrates of 4 to 6 week old culture of P. aeruginosa, the C-9 compound was originally called Pyo Ic and shown to exhibit antibiotic activity (Hays et al. 1945, J. Biol. Chem.,159: 725-749). The name pseudan was applied later (Ritter and Luckner, 1971, Eur. J. Biochem. 18: 391-400), and is the name we use here. The pseudans have been identified as having alkyl chains of C₇-C₁₂, e.g. pseudan IX (Herbert, 1989, 197. The Biosyntheses of Secondary Metabolites. 2nd ed. Chapman and Hall. New York).

The present invention relates to all aspects of 4-hydroxy-2-alkylquinolines which are capable of binding to iron atoms. The 4-hydroxy-2-alkylquinoline can be used in any method or process in which an iron-chelating activity is useful, including, e.g., depleting a liquid source of iron atoms, and treating a living host who has an excess or overload of iron. The present invention also relates to methods and processes for synthesizing 4-hydroxy-2-alkylquinolines.

Although iron is biologically necessary to maintain the health and vitality of living organisms, in excess it can have deleterious effects. There is a need for iron-binding agents which are capable of removing, depleting, etc., surplus iron from hosts suffering from excess or iron overload.

When the amount of free iron exceeds the iron-binding capacity of the blood (e.g., as mediated through the iron-binding protein transferrin), a number of toxic effects are observed. These include, for instance, vasculature damage, increased permeability and fluid loss through the vasculature, mitochondrial damage, lipid peroxidation, renal, tubular, and hepatic necrosis, and ulceration of the stomach and small bowel. See, e.g., Harrison's Principles of Internal Medicine, 12^{th} Edition, Volume 2, McGraw-Hill, 1991, Pages 2174-2175.

There are also a number of disorders which result in excessive iron in the body. For instance, hemochromatosis is an iron-storage disease in which an abnormal increase in the levels of intestinal iron results in its deposition in major organs, such as liver, pancreas, heart, and pituitary, producing tissue damage and impaired function. Without treatment, death may occur from cirrhosis, primary liver cancer, diabetes, or cardiomyopathy. One of the causes of genetic hemochromatosis is a mutation in the HFE gene. See, e.g., Hansen et al., Am. J. Epidemiol, 154(3):193-206, 2001. There are other forms of inherited hemochromatosis. See, e.g., Roy and Andrews, Hum. Mol. Genet., 10(20):2181-6, 2001. Additionally, there is acquired hemochromatosis which is an iron overload disorder that occurs secondarily to other diseases. For example, thalassemia (major and minor) and sideroblastic anemia can result in massive deposits of iron, causing tissue damage and organ failure. Recently, it has been recognized excess body iron is associated with glucose intolerance, type 2 diabetes, gestational diabetes, insulin resistance, and even inflammatory lesions and free radical damage. See, e.g., Fernandez-Real, Diabetes, 51(8):2348-2354, 2002.

In accordance with the present invention, 4-hydroxy-2-alkylquinolines can utilized to treat iron excess, iron, toxicity and iron overload associated with iron poisoning and iron disorders, such as hemochromatosis, thalassemia, diabetes, glucose metabolic disorders, and any conditions or disorders as mentioned above. Various alkylquinolines can be used, e.g., 4-hydroxy-2-C₄-C₁₅-quinolines, 4-hydroxy-2-C₇-C₁₂-quinolines, 4-hydroxy-2-nonylquinoline, etc., with saturated and unsaturated side-chains. In addition, the molecule (e.g., the alkyl side-chain) can be modified, e.g., to chemically conjugate alkylquinolines to substrates for delivery, and pharmaceutical and other uses. Thus, the present invention also relates to derivatives of 4-hydroxy-2-alkylquinolines, especially derivatives that retain iron-binding properties.

4-hydroxy-2-nonylquinoline was originally identified as iron-chelating siderophore in Pseudomonas aeruginosa, although its structure had not been deduced. See, e.g., Royt, Biochim. Biophys. Acta, 939: 493-502, 1988. It had also been isolated from the filtrates of 4 to 6 week old culture of P. aeruginosa and shown to exhibit antibiotic activity (Hays et al., J. Biol. Chem. 159, 725-749, 1945), but its chelating properties went unnoticed. It was later named pseudan (Ritter and Luckner, Eur. J. Biochem. 18, 391-400, 1971).

Any suitable method can be used to prepare 4-hydroxy-2-alkylquinolines. For instance, they can be isolated from natural sources, such as P. aeruginosa and other siderophore containing bacteria using conventional methods, e.g., electrophoresis, mass spectrometry, liquid chromatography, HPLC, thin-layer liquid chromatography, detergent extraction (e.g., non-ionic detergent, Triton X-100, CHAPS, octylglucoside, Igepal CA-630), ethanol extraction, ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis, immuno-purification methodology, etc. A method is described in Royt, Biochim. Biophys. Acta., 939, 493-502 and in the examples.

Additionally, 4-hydroxy-2-alkylquinolines can be prepared synthetically. For example, procedures for the synthesis of 4-hydroxy-2-nonylquinoline are described by Wells, J. Biol. Chem., 196:331-340, 1952; and Somanathan and Smith, J. Heterocyclic Chem., 18:1077, 1981. The examples below provide additional synthetic methods which starts with a lower alkyl 4-hydroxyquinoline, such as 4-hydroxy-2-methylquinoline. Reaction of this compound with sodium amide in liquid ammonia solvent gave the dianion where both aromatic substituents were deprotonated. When the dianion was further reacted with a limited amount of an alkyl halide, selective alkylation was achieved at the 2 methyl carbanion. The desired 4-hydroxy-2-alkylquinolines can be prepared upon work-up with acid.

Thus, the present invention relates to methods of preparing a 4-hydroxy-2-alkylquinolines, comprising, e.g., one or more of the following steps, reacting a 4-hydroxy-lower alkylquinoline, or derivatives thereof, with a strong base in a solvent to produce an anion (e.g., a dianion), and reacting said anion with an alkylating agent to produce a 4-hydroxy-2-alkylquinoline after work-up, e.g., with an acid. A lower alkyl is an alkyl having C1-C3 carbons, preferably C1 or methyl. Any suitable strong base can be used, including, e.g., amide salts (e.g., sodium amide, lithium diisopropylamide), alkyl(aryl)lithiums (e.g., n-butyllithium, t-butyllithium, phenyllithium, etc.), etc. A solvent is selected which is compatible with the base, e.g., liquid ammonia, when an sodium amide salt is utilized, a hydrocarbon solvent or even tetrahydrofuran, with an alkyl(aryl)lithium or LDA is utilized. An alkylating agent, includes any alkyl compound having a good leaving group, such as alkyl halides, alkyl tosylates, and alkyl triflates.

Derivatives of 4-hydroxy-2-alkylquinoline can also be used, especially derivatives comprising a protecting group on the 4-hydroxy position. For example, the 4-hydroxy-lower alkyl-quinoline can contain a protecting group on the 4-position to modify the reactivity of the hydroxyl group in the quinoline. After reacting with a strong base, followed by the alkylation and work-up with an acid, the protecting group can be removed, e.g.. by mild acid hydrolysis. Any suitable protecting group can be used, e.g., t-butyl (O—C₄H₉).

The present invention also relates to pharmaceutical compositions comprising an effective amount of a 4-hydroxy-2-alkylquinoline and a pharmaceutically acceptable carrier. Such pharmaceutical compositions are useful to treat, e.g., iron toxicity, hemochromatosis, thalassemia, sideroblastic anemia, glucose intolerance, diabetes, type 2 diabetes, gestational diabetes, insulin resistance, glucose metabolic disorders, free radical injury, inflammatory lesions, etc, and other disorders associated with excess iron.

These compositions can be in any form in which 4-hydroxy-2-alkylquinoline is capable of binding to iron atoms. For example, it can be isolated, in a membrane (e.g., a phospholipid membrane fragment), in a liposome, in a pro-form which is activated upon contact with the appropriate environment, in a liquid, solid, suspension, colloid, or tablet, etc. By the phrase, “pharmaceutically acceptable carriers,” it is meant any pharmaceutical carrier, such as the standard carriers described, e.g., Remington's Pharmaceutical Science, Eighteenth Edition, Mack Publishing Company, 1990. By “isolated,” it is meant that the compound is essentially free of the components in which it is found with in nature.

The 4-hydroxy-2-alkyl compound is present in the composition in a form in which it is capable of binding iron when the composition is contacted with an iron-containing medium. A property of the quinolines of the present invention is they possess the ability to bind or attach to iron. The binding property indicates that iron attaches specifically to a functional moiety of the 4-hydroxy-2-alkylquinoline. This can also be called “chelation,” since it involves the process of forming bonds between a metallic ion and an organic compound. By being present “in a form in which is capable of binding iron,” it means that the 4-hydroxy-2-alkyl is available to chelate iron. For example, if the 4-hydroxy-2-alkylquinoline is present in a liposome, and the liposome was contacted with blood which contained an excess of iron, the 4-hydroxy-2-alkylquinoline would be able to chelate the excess iron, depleting the blood of iron.

By the phrase “effective amount” as used herein, it is meant an amount of the composition which is adequate to accomplish the desired purpose. Effective amounts can be determined conventionally, e.g., by gradually increasing dosages until the desired effect is achieved, by in vivo or in vitro studies in which iron depletion is monitored, etc.

As mentioned, a 4-hydroxy-2-alkylquinoline can be in any form that is effective for it to chelate iron. It can be associated with any polymer or material in any arrangement that maintains its iron-binding properties. For example, it can be associated with hydrophobic carriers, hydrophobic resins, nanoparticles, hydrophobic insoluble polymers (e.g., copolymers of divinylbenzene and copolymerizable monomer such as styrene, acrylic ester, methacrylic ester, and methacrylic ester resins, acrylic ester resins, polypropylenes, nylons and phenolic resins), polyisobutylcyanoacrylate, polyisohexylcyannoacrylate, polystyrene carboxyl resin, ceramides, steroids, diglycerides, lipids, phospholipids, fatty acids such as stearic acid, gels, emulsions, etc. The association between the active agent and the carrier can be of any suitable type, including, e.g., covalent, noncovalent, hydrophobic, conjugated, immobilized, coated, chemically-bonded (e.g., through linker or spacer groups), etc. When the agent is to be administered to a living subject (e.g., a human patient), the carrier can be physiologically-compatible. However, if its use is ex vivo or for assay use, physiological compatibility may not be a concern.

A 4-hydroxy-2-alkylquinoline, such as 4-hydroxy-2-nonylquinoline, can be associated with a membrane, e.g., a phospholipid bilayer, or with a membrane of a liposome. By the phrase “associated with the membrane of a liposome,” it is mean that the compound is in contact with the lipids which comprise the liposome membrane. Contact with the lipid constituents can be achieved directly or indirectly. For instance, the compound can be associated with the lipid membrane by being incorporated into it, integrated into it, embedded in it (e.g., in the same way lipids are incorporated into lipid bilayers), anchored by its hydrophobic chain, conjugated to it, etc. It can be covalently or noncovalently (e.g., by hydrophobic interactions) anchored to the surface of the liposome. Any association with the lipids can be used.

Phospholipid bilayers, e.g., liposomes, can be prepared by any suitable method. The preparation of liposomes, per se, is not a part of the present invention, since liposomes are well known in the prior art. In general, liposomes have been made by a number of different techniques including, e.g., ethanol injection (e.g., Batzri et al., Biochem. Biophys. Acta. 298 1015, 1973); ether infusion (e.g., Deamer et al., Biochem. Biophys. Acta. 443:629, 1976; and Schieren et al., Biochem. Biophys. Acta. 542:137, 1978), detergent removal (e.g., Razin, Biochem. Biophys. Acta. 265:241, 1972), solvent evaporation, (Matsumato et al., J. Colloid Interface Sci., 62:149, 1977), evaporation of organic solvents from chloroform in water emulsions (REV's) (e.g., Szoka et al., Proc. Natl. Acad. Sci. USA, 75:4194, 1978); extrusions of MLV's or EUV's through a nucleopore polycarbonate membrane (e.g., Olson et al., Biochem. Biophys. Acta. 557:9, 1979); and freezing and thawing of phosopholipid mixtures (e.g., Pick, Archives of Biochem. and Biophysics, 212:186, 1981).

By convention, liposomes are categorized by size, and a 3-letter acronym is used to designate the type of liposome being discussed. Multilamellar vesicles are generally designated “MLV.” Small unilamellar vesicles are designated “SUV,” and large unilamellar vesicles are designated “LUV.” These designations are sometimes followed by the chemical composition of the liposome. For a discussion of nomenclature and a summary of known types of liposomes, see, e.g., Papahadjopoulos, Ann. N.Y. Acad. Sci., 308:1 (1978) and Ann. Repts. Med. Chem., 14:250 (1979).

4-hydroxy-2-alkylquinolines of the present invention can be covalently attached to the surface of pre-formed liposome, or incorporated into its phosopholipid bilayer. The latter can be achieved using an effective method. Reconstitution of a 4-hydroxy-2-alkylquinoline into a bilayer membrane, in such a way that it still retains its iron-binding properties and is exposed on the outer surface of the liposome can be achieved by preparing the liposome in the presence of the compound. Very generally, compounds of the present invention can be mixed with lipid-forming phospholipids, dried down, and then reconstituted with buffer. When sonicated, the resultant mixture is converted into liposomes.

Various procedures can be used. For example, cochleate intermediates can be formed and then converted to phospholipid vesicles. These are methods are described in U.S. Pat. No. 4,871,488. There are several known procedures for making such cochleates. One such method is the so-called standard cochleate obtained by use of the calcium-EDTA-chelation technique described by Papahadjopoulos et al., supra. In this method, a 4-hydroxy-2-alkylquinoline can be mixed with negatively charged phospholipids, such as phosphatidylserine, phosphatidic acid or phosphatidyl glycerol in the absence or presence of cholesterol (up to 3:1, preferably 9:1 w/w) to produce a suspension of multilamellar protein lipid vesicles. These can be converted to small unilamellar protein lipid vesicles by sonication under nitrogen. The resulting vesicles can be dialyzed at room temperature against buffered calcium chloride resulting in the formation of an insoluble precipitate referred to as a cochleate cylinder. After centrifugation, the resulting pellet can be taken up in buffer to yield a “standard” cochleate solution.

A “DC” cochleate can be formed by mixing an amount of phosphotidylserine and cholesterol (e.g., in a 3:1 ratio, and equal to from about 1 to 10 times the weight, preferably equal to four times the weight of the 4-hydroxy-2-alkyquinoline) to prepare the cochleates. Isolated 4-hydroxy-2-alkyquinoline can then be added and vortexed. This solution can then dialyzed against buffered calcium chloride to produce a precipitate which can be called a DC (for direct calcium dialysis) cochleate.

The formation of vesicles from the above intermediates can be carried out by several different methods. In one procedure, the aforesaid cochleates can be pelleted by centrifugation at 60,000×g at about 5° C. The pellets can be re-suspended in a desired buffer by vortexing. Five microliter aliquots of 150 mM EDTA (pH 9.5) can be added with gentle mixing and frequent monitoring of pH. It is desired to maintain pH to near neutrality during the vesicle formation procedure. When the pH became slightly basic, 150 mM EDTA (pH 7.5) was added until the cochleates were dissolved and an opalescent suspension of vesicles was obtained.

Rotary dialysis can also be employed. In this procedure, the supernatants can be removed and transferred to a small segment of dialysis tubing. Small aliquots of buffer (5 to 10 microliters) can be used to rise out the tube and quantitatively tranfer the cochleates to the dialysis bag. The samples can then be dialyzed by rotating at room temperature against buffered 10 mM EDTA (final pH 7.4) until the cochleate precipitate dissolves and an opalescent suspension of vesicles is obtained.

Other methods of reconstituting compounds of the present invention into liposome and other phospholipid bilayers, e.g., U.S. Pat. No. 6,040,167 and 5,709,879. To determine whether the compound has been incorporated into the bilayer membrane in biologically-active form (i.e., able to chelate iron), an iron-binding assay can be carried out as described in the attached Appendix. The amount of quinoline present in the membrane can be adjusted, e.g., by increasing the amount utilized during liposome preparation. The 4-hydroxy-2-alkylquinoline can be anchored by its alkyl tail into the lipid membrane, but other arrangements are possible, as long as activity is retained.

The present invention also relates to using 4-hydroxy-2-alkylquinoline to remove or deplete iron from a liquid. For example, the present invention relates to methods of treating iron excess in a host in need thereof comprising, e.g., administering an effective amount of a composition, such as a pharmaceutical composition comprising an effective amount of a 4-hydroxy-2-alkylquinoline and a pharmaceutically-acceptable carrier, or a liposome comprising a pharmaceutically-effective amount of a 4-hydroxy-2-alkylquinoline which is associated with the membrane of said liposome. As discussed above, any host having excess iron can be treated, including, e.g., hosts suffering from acute iron poisoning or toxicity, hemochromatosis, thalassemia major, sideroblastic anemia, auto-immune haemolytic anaemia, chronic anaemia, idopathic haemochromatosis, iron overload associated with porphyria cutanea tarda, glucose intolerance, type 2 diabetes, gestational diabetes, insulin resistance, inflammatory lesions, free radical damage, including any disorders that are treated with Desferal.

Iron excess indicates that the host contains an amount of iron which is more than the amount that the body normally needs. Removing excess iron, as discussed above, can be useful for treating iron overload diseases, and disorders associated with excessive iron, such as glucose intolerance, type 2 diabetes, gestational diabetes, insulin resistance, inflammatory lesions, and free radical damage. As discussed in more detail below, compositions can be administered in any manner which is effective for depleting the excess iron from the body. In addition to administration, the depletion can also be performed ex vivo.

In addition, 4-hydroxy-2-alkylquinolines can also be used to assay for iron in medium. As discussed below, it changes color upon iron binding, and this change can be measured spectrophotometrically to detect and/or quantitate the amount of iron present in a sample. As already discussed, the 4-hydroxy-2-alkylquinolines must be in a form in which it retains its iron binding properties, e.g., liposomes.

The compositions can be administered by any effective route, e.g., oral, parenteral, enteral, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. It can be administered alone, or in combination with any ingredient(s), active or inactive.

Compositions of the present invention can comprise any amount of which is effective to treat or prevent an iron overload disorder.. The precise dosages administered to a subject depends on a variety of different factors, including, e.g., gender, age, weight, health, stage and grade of the disease, immune status, existence of other health conditions and disorders, body fat, activity status (e.g., whether the individual exercises regularly), diet, other medications and food supplements regularly taken by the subject, hormonal status (e.g., menopausal or pregnant), etc. For these reasons, effective dosages may be empirically determined.

Compositions of the present invention can be administered analogously to deferoxamine (Desferal), another iron-chelating drug. Desferal is administered by subcutaneous or intravenous infusion by a small portable pump. Typically the patient inserts a subcutaneous needle and wears the pump for 9-12 hours each day, usually at night while sleeping. Severely iron overloaded patients may need a continuous infusion through an indwelling central venous catheter. A composition comprising a 4-hydroxy-2-alkylquinoline can be administered in such a way, or, e.g., orally at intervals of about every 2-12 hours.

The compositions can be used to remove iron from any desired source. Such a source can be, e.g., a body fluid, such a whole blood or serum, or any fluid. Any amount of iron can be removed from the source, e.g., 1%, 5%, 10%, 25%, 50%, 80%, 90%, substantially all, etc. Iron removal can be accomplished in as many steps as desired.

### EXAMPLES

Bacteria and culture conditions. Pseudomonas aeruginosa ATCC 15692 was used in all experiments. Cells were grown in tryptic soy broth (DIFCO Laboratories) or in succinate synthetic medium (SSM) as described by Meyer and Abdallah (Meyer and Abdallab, 1978. J. Gen. Microbiol. 107: 319-328). One liter of SSM contains 4 g succinic acid, 1 g (NH₄)₂SO₄, 0.2 g MgSO₄.H₂0, 6 g K₂HPO₄, and 3 g KH₂PO₄ per liter deionized water. The pH was adjusted to 7.0. After sterilization, the medium was supplemented with iron to 30 μM using FeCl₃. A one liter aliquot of media was inoculated with 1 ml mid-logarithmic phase culture, and the culture shaken for 40 hr at 150 rpm at 30° C. in a New Brunswick rotatory shaker. Upon harvesting, cells were washed two times with 0.1 M Tris-HCl (pH 7.8) (Tris-HCl buffer). When indicated, ⁵⁵FeCl₃, 20 μCi, (New England Nuclear) was added to 500 ml SSM.

Membrane preparation and chelator extraction. Cells were harvested and washed two times with cold Tris-HCl buffer. The cells of one liter of media were resuspended in 10 ml Tris-HCl buffer containing 2 mg each of DNase, RNase, and MgCl₂, and the suspension passed one time through a precooled Carver pressure cell (Fred S. Carver Inc., Menomonee Falls, Wis.) at 20,000 lb/in². The membranes were collected by centrifugation at 38,000×g for 1 hr, and washed twice with Tris-HCl buffer. Ten ml ethanol was added to the membranes of cells of one liter of culture. The suspension was mixed and incubated at room temperature for one hr with occasional mixing. Following centrifugation at 38,000×g for 45 min, the ethanol extract was removed.

Purification of the chelator. In earlier studies, the ethanol extract was applied to a silica gel G plate that was then developed using chloroform/ethanol/acetic acid (90:5:5, v/v) (Royt, 1988). The underrated chelator was identified as a yellow band (R_{f} 0.60) on the plate. A purple band (R_{f} 0.77) and brown band (R_{f} 0.85) on a chromatogram were identified as ferrated forms of the chelator. These were eluted from the matrix with ethanol.

In the present study, a Waters 600E HPLC system controlled by Millenium software was used to purify the chelator. Following centrifugation at 137,000×g for 1 hr, the ethanol extract was applied to a preparative C-18 reverse phase HPLC column (250×10 mm, 5 micron, 100 A, Columbus. Phenomonex). The injection volume was 0.5 ml. The mobile phase, containing water (A) and acetonitrile (B), consisted of a linear gradient of initial concentration of 60% A/40% B to 100% B over 40 min at 2 ml/min. The detector wavelength was set at 247.5 nm, and the samples scanned from 190 to 600 nm. Peaks of the chelator were determined by comparing the UV/Vis spectra with those of the brown and purple spots scraped and eluted from the thin layer plate. The solvent from HPLC fractions was removed under vacuum, and the residue dissolved in appropriate solvent. The HPLC-purified chelator was analyzed by FAB mass spectroscopy and ¹³C NMR.

Incorporation of ⁵⁵Fe into the chelator. The ethanol extract of membranes of cells grown in the presence of ⁵⁵FeCl₃ was reduced to half volume with a nitrogen bubbler. Following refrigeration at −20° C., 0.5 ml of the extract was applied to a LH-20 column (1 cm×13 cm) equilibrated with ethanol. Fractions, 350 μl, were collected. The chelator was identified by its UV/Vis spectrum, and its absorbance at 240 nm determined. The ⁵⁵Fe content of each fraction was determined by scintillation counting.

NMR Spectroscopy. NMR spectra were collected in chloroform-d and referenced to internal tetramethylsilane at 0 ppm for ¹³C, on a Bruker DPX300 instrument, at 75 MHz. Polarization transfer sequences (DEPT 90 and DEPT 135) were used for ¹³C assignments.

FAB Mass Spectroscopy. FAB mass spectra were obtained on a JEOL SX102 mass spectrometer operated at an accelerating voltage of 10 kV. Samples were desorbed from a nitrobenzyl alcohol matrix using 6 keV xenon atoms. Mass measurements were performed at 10,000 resolution using electric field scans and the sample peak bracketed by two polyethylene glycol reference ions. Linked scan analysis of the parent ion (MH⁺) was performed to positively identify the compound.

Synthesis of the chelator. Pseudan IX was synthesized from 4-hydroxy-2-methylquinoline via treatment with two equivalents of sodium amide at −33° C., followed by one equivalent of 1-bromooctane, with an aqueous workup to protonate the quinolinolate oxygen.

Micelle formation. Micelles were observed in a condensed ethanol extract of membranes by brightfield microscopy. Also, synthesized pseudan IX, 0.2 M in ethanol, was mixed with 0.2 M FeCl₃ in ethanol in a 3/1 ratio. Following reduction to half the volume using a nitrogen bubbler, micelles were observed as above. An Olympus BX-60 microscope and an Optronics DEI 750 camera were used.

Results

An HPLC chromatogram of the elution of the chelator from the reverse phase column shows a major peak at 33.2 min, compound 1, co-eluting with the brown compound isolated from the TLC plate. The peak at 26.6 min, compound 2, co-eluted with the purple compound seen on the TLC plates. Minor peaks with retention times greater than 33.2 min are also seen on the chromatogram.

The UV/Vis spectra of compounds 1 and 2 eluted from the HPLC column, reveals strong absorption in the UV, and minimal detectable absorption in the visible. The doublet at 315 and 327 nm is characteristic of these compounds. Compound 1 has more absorption at 259 nm and 267 nm than does compound 2. The UV/Vis spectra of the minor peaks eluted from the HPLC before and after 33.2 min are similar to those of compounds 1 and 2. The absorption of those peaks increases at ˜260 and ˜270 nm as does time of elution.

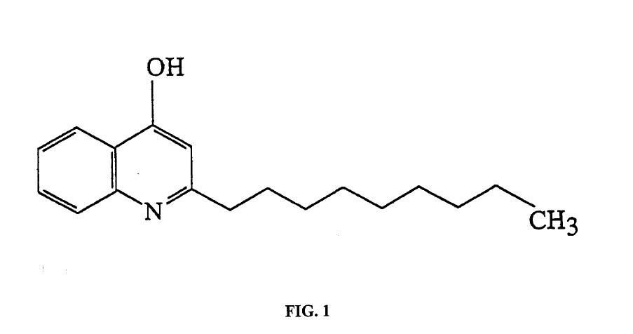

Preliminary mass spectroscopy and proton NMR data suggested that compounds 1 and 2 were 4-hydroxy-2-alkyl quinolines (trivial name, pseudan). In order to confirm this, we synthesized 4-hydroxy-2-nonyl quinoline (pseudan IX, FIG. 1) and compared its properties to those of compounds 1 and 2. Its ¹³C NMR spectrum, FAB mass spectrum, and UV spectrum were the same as those of the compound purified from the bacterium, indicating the two are identical.

The synthesis of 4-hydroxy-2-nonyl quinoline has been reported previously (Wells, 1952, J. Biol. Chem. 196: 331-340). That synthesis appeared to be low yielding and indirect, so a new pathway was devised. This method started with the commercially available (Aldrich) 4-hydroxy-2-methylquinoline. Reaction of this compound with two equivalents of sodium amide in liquid ammonia solvent gave the dianion where both aromatic substituents were deprotonated. Reaction of the dianion with one equivalent of the appropriate alkyl halide gave selective alkylation at the 2 methyl carbanion. The desired 4-hydroxy-2-alkylquinolines are thus prepared upon work-up with acid.

Following exposure to −20° C., the condensed ethanol extract of cells is bilayered, the lower layer red, and the upper layer pale yellow. Visible, macroscopically and microscopically, in the lower layer are pink micelles. Pink micelles, albeit smaller, are also evident upon mixing synthesized pseudan IX with FeCl₃. Application of the lower red layer in the ethanol extract of membranes to a LH-20 column resulted in two bands, an orange band followed by a red band. The chelator, identified by its UV/Vis spectrum, eluted with the ⁵⁵Fe, indicating its iron-binding capacity.

Without further elaboration, it is believed that one skilled in the art can, using the preceding description, utilize the present invention to its fullest extent. The preceding preferred specific embodiments are, therefore, to be construed as merely illustrative, and not limiting of the remainder of the disclosure in any way whatsoever. The entire disclosure of all applications and publications, including U.S. Provisional Application No. 60/331,199, filed Nov. 9, 2001, and Royt et al., Bioorg Chem., 2001 Dec., 29(6):387-97, are hereby incorporated by reference in their entirety.

1. A pharmaceutical composition comprising an effective amount of a 4-hydroxy-2-alkylquinoline, or derivative thereof, and a pharmaceutically-acceptable carrier.

2. A pharmaceutical composition of claim 1, wherein the 4-hydroxy-2-alkylquinoline is 4-hydroxy-2-nonylquinoline.

3. A pharmaceutical composition of claim 1, wherein said 4-hydroxy-2-alkylquinoline is associated with the membrane of a liposome.

4. A pharmaceutical composition of claim 1, wherein said 4-hydroxy-2-alkylquinoline is capable of binding iron when said composition is contacted with an iron-containing medium.

5. A pharmaceutical composition of claim 1, wherein said 4-hydroxy-2-alkylquinoline is capable of binding iron when said liposome is contacted with an iron-containing medium.

6. A liposome comprising a pharmaceutically-effective amount of a 4-hydroxy-2-alkylquinoline which is associated with the membrane of said liposome.

7. A liposome of claim 6, wherein the 4-hydroxy-2-alkylquinoline is 4-hydroxy-2-nonylquinoline.

8. A liposome of claim 6, wherein said 4-hydroxy-2-alkylquinoline is embedded in the membrane of said liposome.

9. A liposome of claim 6, wherein said 4-hydroxy-2-alkylquinoline is capable of binding iron when said composition is contacted with an iron-containing medium.

10. A liposome of claim 6, wherein the liposome comprises phospholipids.

11. A liposome of claim 6, wherein the liposome comprises phosphatidylcholine.

12. A method of treating iron accumulation in a host in need thereof comprising,
administering an effective amount of a composition of claim 1.

13. A method of claim 12, wherein said host has thalassemia, glucose intolerance, type 2 diabetes, or gestational diabetes.

14. A method of treating iron accumulation in a host in need thereof comprising,
administering an effective amount of a composition of claim 6.

15. A method of claim 14, wherein said host has thalassemia, glucose intolerance, type 2 diabetes, or gestational diabetes.

16. A method of removing iron from a liquid comprising,
contacting a liquid containing iron with an effective amount of an isolated 4-hydroxy-2-alkylquinoline, under conditions effective for said 4-hydroxy-2-alkylquinoline to bind to iron in said liquid, and
separating said 4-hydroxy-2-alkylquinoline from said liquid, whereby the amount of iron present in said liquid is less than the amount prior to said contact.

17. A method of claim 16, wherein said liquid is aqueous.

18. A method of preparing a 4-hydroxy-2-alkylquinoline, comprising,
reacting a 4-hydroxy-lower alkyl-quinoline, or derivative thereof, with a strong base in a solvent to produce an anion,
and reacting said anion with an alkylating agent to produce a 4-hydroxy-2-alkylquinoline.

19. A method of claim 18, further comprising treating with an acid to produce said 4-hydroxy-2-alkylquinoline.

20. A method of claim 18, wherein said derivative comprises a hydroxyl-protecting group.
